(19) United States
(12) Patent Application Publication
(10) Pub. No.: US 2009/0287235 A1
(43) Pub. Date: Nov. 19, 2009

(54) Cutting Assembly For Use In A Laparoscopic Cutting Instrument

(76) Inventors: Richard Fortier, Concord, MA (US); Richard Wisdom, Hyde Park, MA (US)

Correspondence Address: Tyco Healthcare Group LP, 60 MIDDLETOWN AVENUE, NORTH HAVEN, CT, 06473, US

(21) Appl. No.: 12/436,179

(22) Filed: May 6, 2009

Related U.S. Application Data
(60) Provisional application No. 61/053,136, filed on May 14, 2008.

(51) Int. Cl. A61B 17/3201

(52) U.S. Cl. 606/174

(57) ABSTRACT

An improved cutting assembly for use with a surgical instrument is provided and includes a pair of cutting blades pivotally mounted on a yoke and a drive member engageable with the cutting blades to move the cutting blades between an open position substantially spaced apart from each other to a closed position wherein the cutting blades are in close cooperative alignment to cut tissue. One or more biasing members are provided adjacent the cutting blades to prevent the cutting blades from splaying or separating during the cutting of tissue.

### BACKGROUND

1. Technical Field

The present disclosure relates to an improved cutting assembly for use in a laparoscopic cutting instrument. More particularly, the present disclosure relates to an improved cutting assembly incorporating biasing members to maintain cutting blades of the cutting assembly in close cooperative alignment during the cutting of tissue.

2. Background of Related Art

Various surgical instruments are used to cut or shear tissue. The instruments generally include a pair of cutting blades each having sharp tissue engaging edges for cutting tissue. Actuation devices are provided for moving the cutting blades between an open position substantially spaced apart from each other to a closed position wherein the cutting blades are in close cooperative alignment to cut tissue.

When the cutting blades engage the tissue with only distal sections of cutting portions of the cutting blades, the distal sections may be forced apart resulting in splaying of the cutting blades such that the cutting blades form an angle relative to each other resulting in incomplete or inconsistent cutting of tissue. Likewise, when the cutting portions of the blades engaged the tissue substantially the entire lengths thereof, the cutting blades may be forced apart or separated substantially parallel to each other again resulting in inconsistent or incomplete cutting of tissue.

Therefore, it is desirable to provide an improved cutting assembly incorporating biasing members to maintain the cutting blades in close cooperative alignment during the cutting of tissue.

### SUMMARY

There is disclosed an improved cutting assembly for use with a surgical cutting instrument which generally includes a yoke, a first cutting blade and a second cutting blade pivotally mounted to the yoke. The first cutting blade includes a central portion, a cutting portion extending distally from the central portion and a driving portion extending proximally from the central portion. The second cutting blade also includes a central portion, a cutting portion extending distally from the central portion and a driving portion extending proximally from the central portion. A biasing member is positioned between the driving portions of the first and second cutting blades to prevent splaying of the first and second cutting blades during cutting of tissue.

A drive pin is engageable with the driving portions of the first and second cutting blades to move the cutting portions of the first and second cutting blades between an open position substantially spaced apart to a closed position wherein the cutting portions of the first and second cutting blades are in substantial close cooperative alignment to cut tissue.

In one embodiment, the biasing member is positioned on the drive pin. The driving portions of the first and second cutting blades include drive slots angled relative to each other such that movement of the drive pin within the drive slots moves the first and second cutting blades between the open and closed positions.

In one embodiment, the biasing member is an elastomeric member. In a specific embodiment, the elastomeric member is a polymeric member.

In an alternative embodiment, the biasing member is a spring washer. In one specific embodiment, the spring washer is a wave washer. In another specific embodiment, the spring washer is a belleville washer.

There is also disclosed an improved cutting assembly for use with a surgical cutting instrument including a yoke having a first arm and a second arm and a pivot pin mounted between the first and second arms. First and second cutting blades are provided and are pivotally mounted on the pivot pin. The improved cutting assembly additionally includes a biasing member positioned between the one of the first and second arms and one of the first and second cutting blades. The biasing member prevents separation of the first and second blades substantially parallel to each other during cutting of tissues.

It is also contemplated that the biasing member could be used in surgical shears wherein one of the first and second arms is fixed relative to the other.

There is also disclosed an improved laparoscopic cutting instrument including a handle and a trigger or lever pivotally mounted on the handle. An elongate tubular member extends distally from the handle. A drive rod is provided and has a proximal and distal end, the proximal end of the drive rod being connected to the lever. The improved laparoscopic cutting instrument additionally includes a yoke having a first arm and a second arm, the yoke being mounted on the distal end of the elongate tubular member. First and second cutting blades are pivotally mounted relative to the yoke. The first cutting blade includes a central portion, a cutting portion extending distally from the central portion and a driving portion extending proximally from the central portion. Similarly, the second cutting blade includes a central portion, a cutting portion extending distally from the central portion and a driving portion extending proximally from the central portion. At least one biasing member is provided and is engagable with one of the first and second cutting blades. In one embodiment, a pivot pin is mounted between the first and second arms of the yoke and the at least one biasing member is positioned on the pivot pin. In another embodiment, a drive pin is provided and is engageable with the driving portions of the first and second cutting blades, wherein the at least one biasing member is positioned on the drive pin

### DESCRIPTION OF THE DRAWINGS

An embodiment of the presently disclosed improved cutting assembly is disclosed herein with reference to the drawings, wherein:

FIG. 1 is a side view of a laparoscopic cutting instrument incorporating the disclosed improved cutting assembly;

FIG. 2 is a top view of a prior art cutting assembly;

FIG. 3 is a top view of the prior art cutting assembly with parts separated;

FIG. 4 is a partial side view of blades of the prior art cutting assembly in an open position;

FIG. 5 is a partial side view with the blades of the prior art cutting assembly in a closed position;

FIG. 6 is a top view of the blades of the prior art cutting assembly engaging tissue with a shallow bite;

FIG. 7 is a top view of the blades of the prior art cutting assembly engaging tissue with a deep bite;

FIG. 8 is a top view of the disclosed improved cutting assembly;

FIG. 9 is a top view of the improved cutting assembly with parts separated;

FIG. 10 is a top view of the improved cutting assembly with blades of the improved cutting assembly engaging tissue with a shallow bite; and

FIG. 11 is a top view of the blades of the improved cutting assembly engaging tissue with a deep bite.

### DETAILED DESCRIPTION OF EMBODIMENTS

An embodiment of the presently disclosed improved cutting assembly for use in a laparoscopic cutting instrument will now be described in detail with reference to the drawings wherein like numerals designate identical or corresponding elements in each of the several views. As is common in the art, the term ‘proximal” refers to that part or component closer to the user or operator, i.e. surgeon or physician, while the term “distal” refers to that part or component further away from the user.

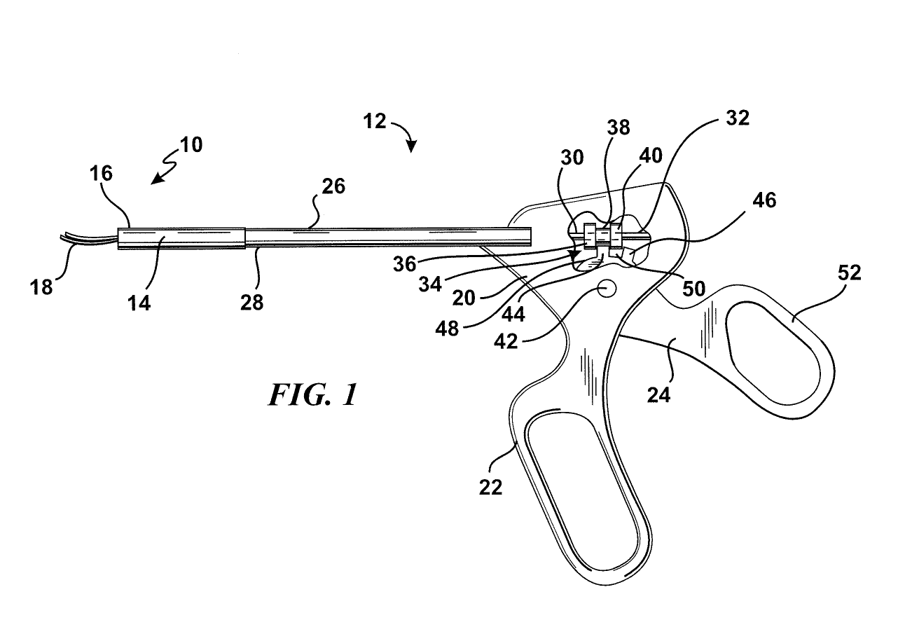

Referring to FIG. 1, there is disclosed an improved cutting assembly 10 for use with a laparoscopic cutting instrument 12. Improved cutting assembly 10 generally includes a yoke 14 having a first blade 16 and a second blade 18 movably mounted thereon. First and second blades 16 and 18 are provided to cut various tissues and are movable from an open position wherein first and second blades 16 and 18 are spaced apart to a closed position wherein first and second blades 16 and 18 are substantially adjacent to each other.

Laparoscopic cutting instrument 12 generally includes a handle 20 having an integral finger grip 22 depending therefrom. A thumb lever 24 is movably mounted on handle 20 and operates to move first and second blades 16 and 18, respectively, between the open and closed positions to cut tissue in a manner described in more detail hereinbelow. An elongate tubular member 26 extends distally from handle 20. Improved cutting assembly 10 is mounted on a distal end 28 of elongate tubular member 26. Specifically, yoke 14 is mounted on distal end 28 of elongate tubular member 26. It should be noted that, while yoke 14 is disclosed as a stand alone component of improved cutting assembly 10, yoke 14 may be formed integrally with elongate tubular member 26.

In order to move first blade and second blade 16 and 18 between the opening closed positions, a drive rod 30 is movably mounted within handle 20 and elongate tubular member 26. Drive rod 30 is movable in the proximal and distal directions and slides over a guide rod 32 fixedly mounted within handle member 20. Guide rod 32 extends substantially through handle 20 and at least partially into elongate tubular member 26. A shuttle 34 is affixed to drive rod 30 and includes a distal collar 36, a central portion 38 and a proximal collar 40. Shuttle 34 is provided to move drive rod 30 between distal and proximal positions and is slideably mounted on guide rod 32.

As noted hereinabove, thumb lever 24 is provided to move first and second blades 16 and 18 between the open and closed positions. Thumb lever 24 is pivotally mounted on handle 20 by a pivot pin 42. In order to move shuttle 34, and thus drive rod 30, distally and proximally within handle 20 and elongate tubular member 26, a pair of sprockets 44 and 46 are formed on a distal end 48 of thumb lever 24 and define a gap 50 therebetween. Proximal collar 40 of shuttle 34 is positioned within gap 50 defined by sprockets 44 and 46. Thus, movement of a thumb grip 52 of thumb lever 24 distally towards finger grip 22 of handle 20 results in proximal movement of shuttle 34 and thus of drive rod 30 within elongate tubular member 26. Likewise, movement of thumb grip 52 to a proximal position relative to finger grip 22 drives shuttle 34, and thus drive rod 30, distally.

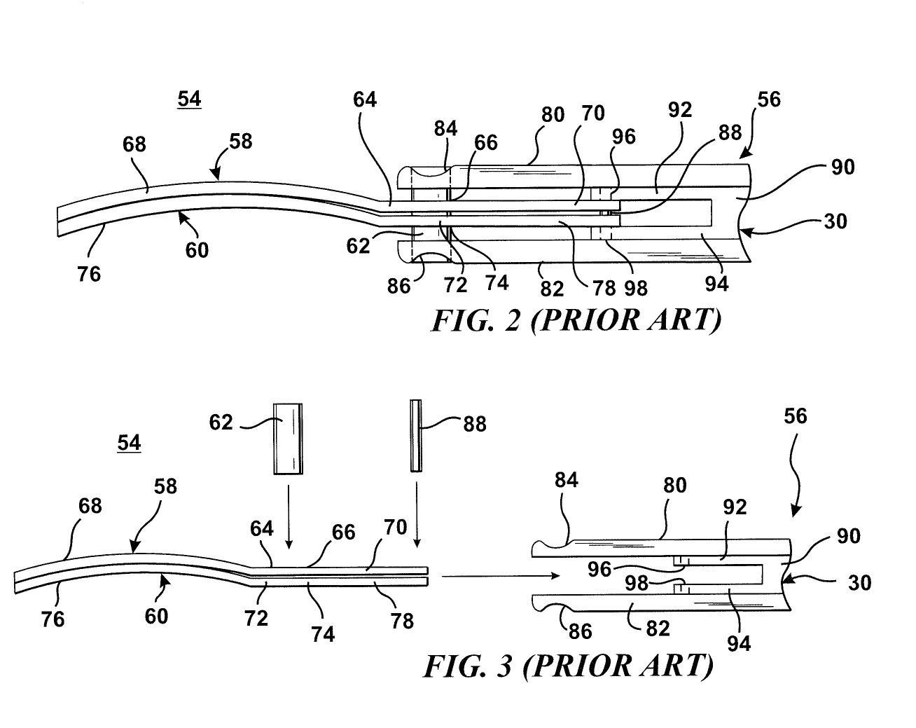

Referring now to FIGS. 2-7, and an initially with respect to FIGS. 2 and 3, there is disclosed a prior art cutting assembly 54 of the type commonly used with laparoscopic cutting instruments. Prior art cutting assembly 54 generally includes a yoke 56 having first and second cutting blades 58 and 60, respectively, movably mounted thereon. As noted with respect to improved cutting assembly 10 described hereinabove, yoke 56 may be formed integrally with an elongate tubular member of a surgical cutting instrument or may be a stand along component of prior art cutting assembly 54. First and second cutting blades 58 and 60 are pivotally mounted on a pivot pin 62 attached to yoke 56. First cutting blade 58 includes a central portion 64, defining a bore 66, a cutting portion 68 extending distally from central portion 64 and a driving portion 70 extending proximally from central portion 64. Pivot pin 62 extends through bore 66 in central portion 64 to pivotally mount first cutting blade 58 to yoke 56. Similarly, second cutting blade 68 includes a central portion 72 defining a bore 74. Second cutting blade 60 is pivotally mounted on yoke 56 by means of pivot pin 62 extending through bore 74. Second cutting blade 68 includes a cutting portion 76 extending distally from central portion 72 and a driving portion 78 extending proximally from central portion 72. Yoke 56 includes distally extending first and second arms 80 and 82 defining pin bores 84 and 86, respectively. Pivot pin 62 extends through bore 84 in first arm 80, through bores 66 and 74 in first and second cutting blades 58 and 60, and through bore 86 in second arm 82. Prior art cutting assembly 54 additionally includes a drive pin 88 which is engageable with driving portion 70 and 78 of first and second cutting blades 58 and 60 to move first and second cutting blades 58 and 60 between the open and closed positions in a manner discussed in more detail hereinbelow.

As noted hereinabove, movement of drive rod 30, in response to actuation of thumb lever 24, operates to open and close the blades of a cutting assembly mounted on surgical instrument 12. A distal end 90 of drive rod 30 includes a first arm 92 and a second arm 94. First and second arms 92 and 94 include respective bores 96 and 98 for receipt of drive rod 88. Distal end 90 of drive rod 30 is longitudinally movable between first and second arms 80 and 82 of yoke 54 to move drive pin 88 within first and second cutting blades 58 and 60 to open and close first and second cutting blades 58 and 60 in a manner described hereinbelow.

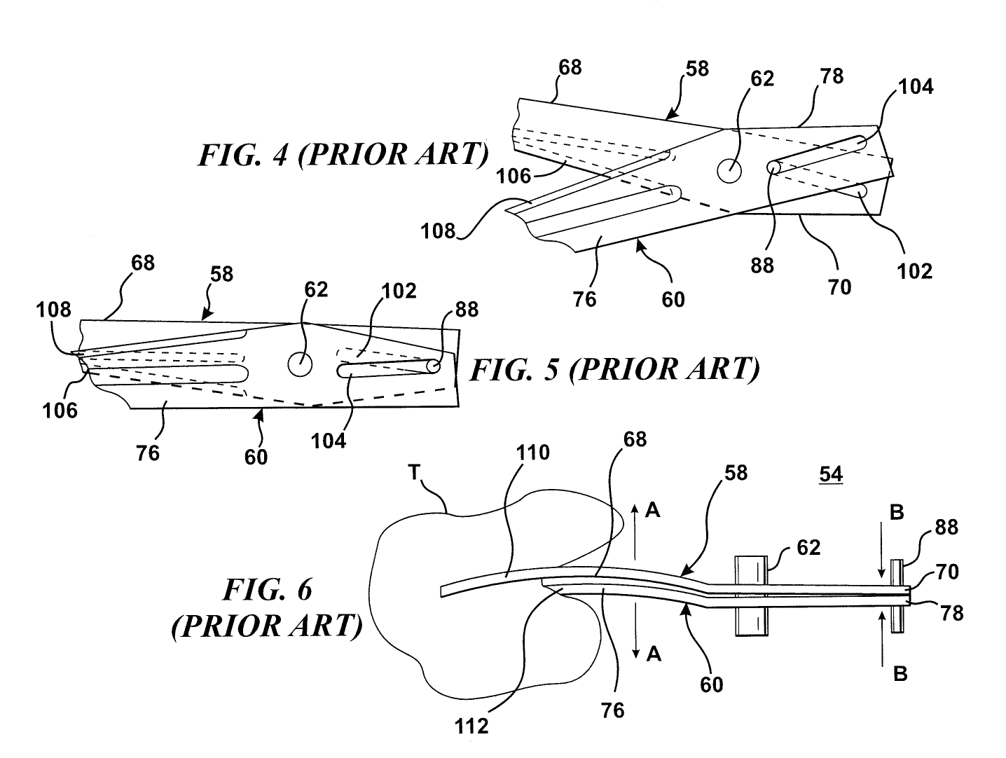

Referring now to FIGS. 4 and 5, in order to move first and second cutting blades 58 and 60 between the open and closed positions, first cutting blade 58 includes a drive slot 102 formed in driving portion 70 and second cutting blade 60 includes a drive slot 104 are formed in driving portion 78. Drive pin 88 extends through drive slots 102 and 104 and is longitudinally movable therein. Drive slots 102 and 104 are formed at angles relative to each other such that distal movement of drive pin 88 through drive slots 102 and 104 moves cutting blades 58 and 60 to an open position wherein cutting portions 68 and 76 are spaced apart from each other (FIG. 4). Likewise, proximal movement of drive pin 88 within drive slots 102 and 104 moves cutting portions 68 and 76 of first and second cutting blades 58 and 60, respectively, to a closed position wherein cutting portions 68 and 76 are substantially adjacent each other (FIG. 5).

As shown in FIGS. 4 and 5, cutting portion 68 of first cutting blade 58 includes a sharp tissue cutting edge 106 and cutting portion 76 of second cutting blade 60 includes a sharp tissue cutting edge 108. Sharp tissue cutting edges 106 and 108 are configured to sever or cut tissue captured therebetween as first and second cutting blades 58 and 60 are moved from the open to the closed position. This is accomplished when first and second sharp tissue cutting edges 106 and 108 are in close cooperative alignment engaging each other as they slice along through the tissue.

Referring to FIG. 6, it should be noted that cutting assembly 54 may be manufactured to certain standards or tolerances resulting in dimensional variations in the sizes or thicknesses of the blades, pins, yoke spacing, etc. This can result in variations in blade interference and contact pressure with tissue T. Thus, in use, when cutting assembly 54 is used to cut a tissue, such as tissue T, taking a shallow “bite” of tissue T by engagement of only distal sections 110 and 112 of cutting portions 68 and 76 with tissue T, distal sections 110 and 112 may be forced apart such that cutting portions 68 and 76 are not in close cooperative align engaging each other and results in an angled separation or “splaying” of cutting portions 68 and 70 in the directions of arrows A. This splaying of cutting portions 68 and 76 may result in an inability of cutting edges 106 and 108 to sever tissue T or may result in an inconsistent cutting or severing of tissue T. As shown, splaying of cutting portions 68 and 76 results in movement of driving portions 70 and 78 inwardly, in the direction of arrows B, towards each other along drive pin 88. This can further have been negative affect of wedging or camming drive pin 88 relative to driving portions 70 and 78 resulting in excess friction and problems in opening and closing cutting blades 58 and 60. Therefore, when cutting assembly 54 is used to take a shallow bite of tissue T, cutting blades 58 and 60 are skewed at an angle relative to each other resulting in incomplete or inconsistent cutting of tissue T.

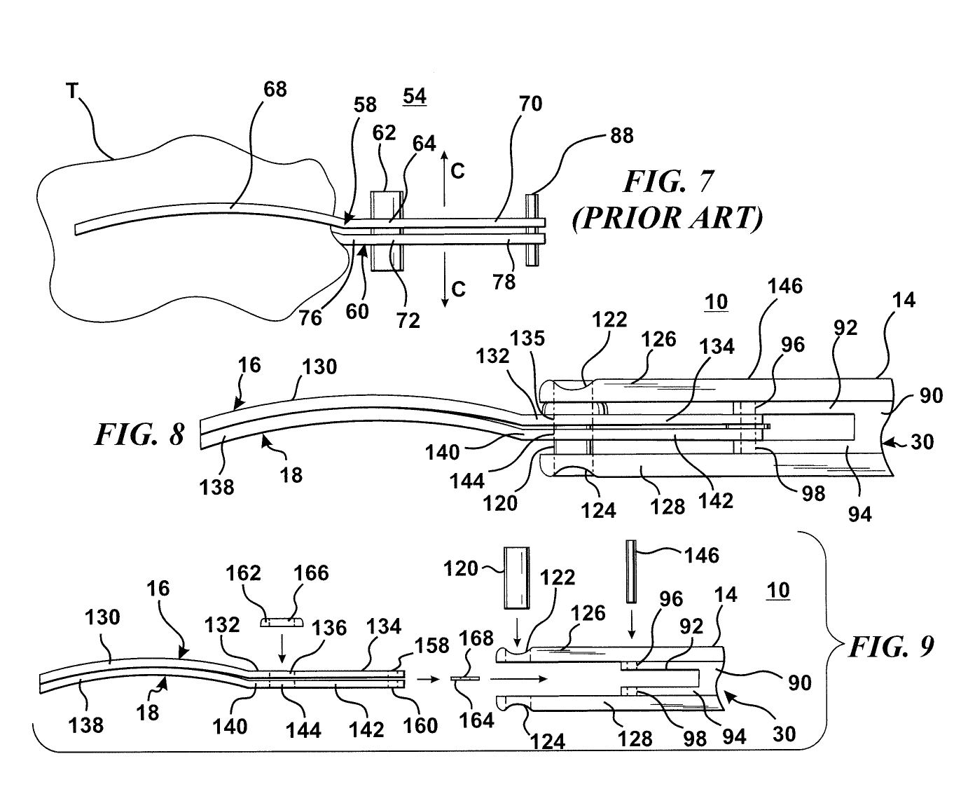

Referring to FIG. 7, in contrast to the procedure disclosed with respect to FIG. 6 hereinabove, when cutting assembly 54 is manipulated to take a deep bite of tissue T and is used to engage tissue T substantially along the length of cutting portions 68 and 76 of cutting blades 58 and 60, respectively, the dimensional differences in the manufacturing tolerances or “looseness” of cutting assembly 54 may result in separation of cutting blades 58 and 60 parallel to each other in the direction of arrows C along substantially the entire length thereof. This also may result in an inability of cutting edges 106 and 108 (not shown) of cutting blades 58 and 62 completely or consistently sever tissue T.

Referring now to FIGS. 8 through 11, and initially with regard to FIGS. 8 and 9, as noted hereinabove improved cutting assembly 10 generally includes yoke 14 and first and second cutting blades 16 and 18 movably mounted on yoke 14. A pivot pin 120 is provided and extends through bores 122 with 124 formed in arms 126 and 128 of yoke 14. First cutting blade 16 includes a cutting portion 130, a central portion 132 and a driving portion 134. A bore 136 is formed through central portion 132. Second cutting blade 18 includes a cutting portion 138, a central portion 140 and a driving portion 142. Second cutting blade 18 also includes a bore 144 formed through central portion 140. First and second cutting blades 16 and 18 are pivotally mounted on yoke 14 by extension of pivot pin 120 through bores 136 and 144 in central portions 132 and 140, respectively.

As noted hereinabove, distal end 90 of drive rod 30 includes first and second arms 92 and 94 defining bores 96 and 98, respectively. Improve cutting assembly 10 includes a drive pin 146 which is positioned through bores 96 and 98 of drive rod 30. While not explicitly shown, driving portions 134 and 142 of first and second cutting blades 16 and 18 are formed with driving slots 158 and 160 similar to driving slots 102 and 104 (FIG. 4) described with respect to cutting blades 58 and 60 of prior art cutting assembly 54 described hereinabove. Thus, movement of drive rod 30 longitudinally within yoke 14 moves drive pin 146 within slots 158 and 160 in driving portions 134 and 142 to open and close first and second cutting blades 16 and 18 substantially in the manner described hereinabove with respect to first and second cutting blades 58 and 60.

Improved cutting assembly 10 is specifically designed to prevent or minimize splaying or separation of cutting blades 16 and 18 when taking shallow and deep bites of tissue. Improved cutting assembly 10 generally includes one or more biasing members, such as first biasing member 162 and second biasing member 164, to maintain first and second cutting blades 16 and 18 in close cooperative alignment while cutting tissue T. In one embodiment, first biasing member 162 includes a bore 166 and is positioned over pivot pin 120. In a specific embodiment, as shown, first biasing member 162 is positioned between one of first and second central portions 132 and 140, of cutting blade 16 and 18 respectively, and arms 126 and 128 of yoke 14. First biasing member 162 maintains consistent pressure on central portions 132 and 140 of cutting blade 16 and 18 to prevent separation of cutting blades 16 and 18 when taking deep bites of tissue T.

Second biasing member 164 is formed with a bore 168 and is positioned about drive pin 146. In a specific embodiment, second biasing member 164 is positioned between driving portions 134 and 142 of first and second cutting blades 16 and 18 to maintain driving portions 134 and 142 in a spaced apart condition and prevent splaying of first and second cutting blades 16 and 18 when taking relatively shallow bites of tissue T.

First and second biasing members 162 and 164 are formed as elastomeric elements in order to maintain pressures against first and second cutting blades 16 and 18. First and second biasing members 162 and 164 may be formed as polymeric members or may take the form of spring washers. Examples of suitable spring washers may include wave washers or, in more specific embodiments, Belleville washers such as those disclosed in U.S. Pat. No. 7,306,059 to Ide, the entire disclosure of which is incorporated by reference herein.

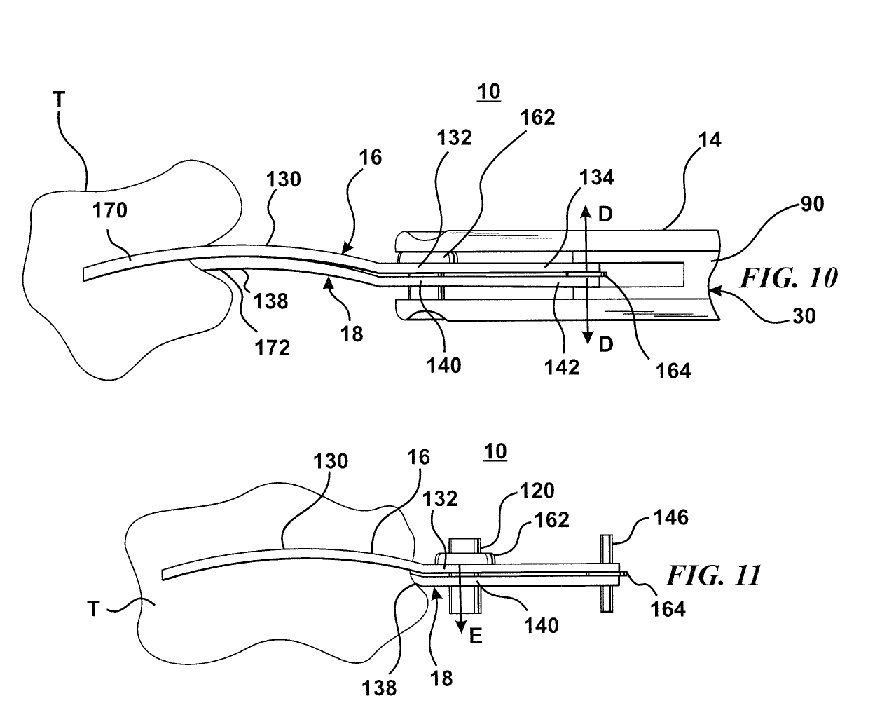

Referring now to FIG. 10, in use, when improves cutting assembly 10 is manipulated such that cutting blades 16 and 18 take a shallow bite of tissue T and engage tissue T along distal sections 170 and 172 of cutting portions 130 and 138, the provision of second biasing member 164 between driving portions 134 and 142 of first and second cutting blades 16 and 18 exerts an outward force in the direction of arrows D on driving portions 134 and 142 to prevent splaying of cutting portions 130 and 138. This outwardly directed force maintains the cutting edges (not shown) of cutting portions 130 and 138 in close cooperative alignment during the cutting of tissue T.

Similarly, as shown in FIG. 11, when improves cutting assembly 10 is manipulated to engage tissue T substantially along the length of cutting portions 130 and 138 of first and second cutting blades 16 and 18, the provision of first biasing member 162 between an arm of yoke 14, such as, for example, arm 126, exerts an inwardly directed force against at least one of central portions 132 and 140, here shown against central portion 132, of first and second cutting blades 16 and 18 to prevent the separation of first and second cutting blades 16 and 18 parallel to each other when taking a deep bite of tissue T thus insuring more complete and consistent cutting of tissue T by the cutting edges of cutting blades 16 and 18.

Thus, the provision of first and second biasing members 162 and 164 in improved cutting assembly 154 aides in preventing the splaying or separation of cutting blades 16 and 18 when taking shallow or deep bites of tissue T.

It will be understood that various modifications may be made to the embodiments disclosed herein. For example, the disclosed biasing member may be provided along other locations of a pivot pin of the cutting blades, such as, for example, in between the cutting blades, etc. Further, more than one biasing member may be provided on the pivot pin or driving pin of the disclosed improved cutting assembly to prevent splaying or separation of the blades during use. Additionally, the disclosed improved laparoscopic cutting assembly may be used in other surgical devices such as, for example, endoscopic biopsy devices, etc. Therefore, the above description should not be construed as limiting, but merely as exemplifications of particular embodiments. Those skilled in the art will envision other modifications within the scope and spirit of the claims appended hereto.

1. An improved cutting assembly for use with a surgical cutting instrument comprising:
a yoke;
a first cutting blade pivotally mounted relative to the yoke, the first cutting blade including a central portion, a cutting portion extending distally from the central portion and a driving portion extending proximally from the central portion;
a second cutting blade pivotally mounted relative to the yoke, the second cutting blade including a central portion, a cutting portion extending distally from the central portion and a driving portion extending proximally from the central portion; and
a biasing member positioned between the driving portions of the first and second cutting blades.

2. The improved cutting assembly as recited in claim 1, further comprising a drive pin engageable with the driving portions of the first and second cutting blades to move the cutting portions of the first and second cutting plates between an open position substantially spaced apart to a closed position wherein the cutting portions of the first and second cutting blades are in substantial close cooperative alignment.

3. The improved cutting assembly as recited in claim 2, wherein the biasing member is positioned on the drive pin.

4. The improved cutting assembly as recited in claim 2, wherein the driving portions of the first and second cutting blades include drive slots angled relative to each other such that movement of the drive pin within the drive slots moves the first and second cutting blades between the open and closed positions.

5. The improved cutting assembly as recited in claim 1, wherein the biasing member is an elastomeric member.

6. The improved cutting assembly as recited in claim 5, wherein the elastomeric member is a polymeric member.

7. The improved cutting assembly as recited in claim 1, wherein the biasing member is a spring washer.

8. The improved cutting assembly as recited in claim 7, wherein the spring washer is a wave washer.

9. The improved cutting assembly as recited in claim 7, wherein the spring washer is a belleville washer.

10. An improved cutting assembly for use with a surgical cutting instrument comprising:
a yoke having a first arm and a second arm;
a pivot pin mounted between the first and second arms;
a first cutting blade pivotally mounted on the pivot pin;
a second cutting blade pivotally mounted on the pivot pin; and
a biasing member positioned between the one of the first and second arms and one of the first and second cutting blades.

11. The improved cutting assembly as recited in claim 10, wherein the biasing member is an elastomeric member.

12. The improved cutting assembly as recited in claim 11, wherein the elastomeric member is a polymeric member.

13. The improved cutting assembly as recited in claim 10, wherein the biasing member is a spring washer.

14. The improved cutting assembly as recited in claim 13, wherein the spring washer is a wave washer.

15. The improved cutting assembly as recited in claim 13, wherein the spring washer is a belleville washer.

16. An improved laparoscopic cutting instrument comprising;
a handle;
a lever pivotally mounted on the handle;
an elongate tubular member extending distally from the handle;
a drive rod having a proximal end and distal end, the proximal end of the drive rod being connected to the lever;
a yoke having a first arm and a second arm, the yoke being mounted on the distal end of the elongate tubular member;
a first cutting blade pivotally mounted relative to the yoke, the first cutting blade including a central portion, a cutting portion extending distally from the central portion and a driving portion extending proximally from the central portion;
a second cutting blade pivotally mounted relative to the yoke, the second cutting blade including a central portion, a cutting portion extending distally from the central portion and a driving portion extending proximally from the central portion; and
at least one biasing member engagable with one of the first and second cutting blades.

17. The improved laparoscopic cutting instrument as recited in claim 16, further comprising a pivot pin mounted between the first and second arms of the yoke, wherein the at least one biasing member is positioned on the pivot pin.

18. The improved laparoscopic cutting instrument as recited in claim 16, further comprising a drive pin engageable with the driving portions of the first and second cutting blades, wherein the at least one biasing member is positioned on the drive pin

19. The improved laparoscopic cutting instrument as recited in claim 16, wherein the biasing member is a wave washer.

20. The improved cutting instrument as recited in claim 16, wherein the biasing member is a belleville washer.
